(19) United States
(12) Patent Application Publication
(10) Pub. No.: US 2017/0100094 A1
(43) Pub. Date: Apr. 13, 2017

(54) OBJECT INFORMATION ACQUIRING APPARATUS AND INFORMATION PROCESSING METHOD

(71) Applicant: CANON KABUSHIKI KAISHA, Tokyo (JP)

(72) Inventor: Hisafumi Ebisawa, Tokyo (JP)

(21) Appl. No.: 15/279,560

(22) Filed: Sep. 29, 2016

(30) Foreign Application Priority Data
Oct. 7, 2015 (JP) 2015-199260

(51) Int. Cl. A61B 8/00; A61B 8/08; G01N 25/00

(52) U.S. Cl. CPC A61B 8/4281; G01N 25/00; A61B 8/0825; A61B 8/40; A61B 8/4209; A61B 8/546; A61B 8/5207

(57) ABSTRACT

An object information acquiring apparatus of the present invention has a conversion element receiving an acoustic wave generated in and propagated from an object and converting the acoustic wave to an electric signal, a holding unit holding an acoustic matching member that acoustically matches the object to the probe, a temperature measuring unit measuring temperatures at a plurality of positions in the acoustic matching member, a reconstruction processing unit acquiring a sound speed of the acoustic matching member and characteristic information of the object based on the sound speed and the electric signal, and a controlling unit performing control based on the temperatures measured at the plurality of positions.

### BACKGROUND OF THE INVENTION

Field of the Invention

The present invention relates to an object information acquiring apparatus and an information processing method.

Description of the Related Art

As an apparatus used in the diagnosis of breast cancer, an X-ray mammography apparatus is widely used which images object information detected by a detector when X-rays are applied. However, because of the problems related to radiation exposure that are inherent to the X-ray mammography apparatus, an imaging apparatus that uses an acoustic wave (typically an ultrasound wave) and is free of radiation exposure is now attracting attention.

In the acoustic wave imaging apparatus, an acoustic matching member for matching the acoustic impedance of an object to that of a probe is required. Herein, in order to image a sound source position in the object with high accuracy, it is necessary to accurately determine the time or distance of propagation of the ultrasound wave. Accordingly, even in an apparatus in which the object is positioned at a distance from the probe, it is necessary to ascertain the sound speed of the acoustic matching member with good precision. Japanese Patent Application Laid-open No. H11(1999)-056834 describes a system in which water and oil as the acoustic matching member are disposed between the object and the probe, the sound speed is ascertained by measuring the temperatures thereof, and image quality degradation is thereby prevented.

Patent Literature 1: Japanese Patent Application Laid-open No. H11(1999)-056834

### SUMMARY OF THE INVENTION

FIG. 13 shows the imaging apparatus of Japanese Patent Application Laid-open No. H11(1999)-056834. The apparatus holds layers of water 1304 and oil 1304 serving as the acoustic matching member between an object 1301 and a probe 1302, using a holding container 1310 and a holding member 1309. The apparatus reconstructs favorable image quality by calculating the sound speed of the acoustic matching member using the result of measurement of a temperature sensor 1305 installed in the acoustic matching member.

However, the temperature in the acoustic matching member is not always uniform. The temperature is particularly influenced by the temperature of the object in the vicinity of the object and by room temperature in the vicinity of the holding container. As a result, a temperature distribution occurs in the acoustic matching member, and a variation of the sound speed occurs due to the temperature distribution. Installation of only one thermometer is not enough for precise acquisition of the variation of the sound speed, resulting in a hindrance in improving the accuracy of the reconstruction. In addition, the influence becomes more significant as the volume of the acoustic matching member becomes larger.

The present invention has been made in view of the above problem. An object of the present invention is to improve accuracy of information acquisition based on temperature information of the acoustic matching member in an apparatus that acquires object information by using the acoustic wave.

The present invention provides an object information acquiring apparatus comprising:

a conversion element configured to receive an acoustic wave that is generated in and propagated from an object and convert the acoustic wave to an electric signal;

a holding unit configured to hold an acoustic matching member that acoustically matches the object to the conversion element;

a temperature measuring unit configured to measure temperatures at a plurality of positions in the acoustic matching member;

a reconstruction processing unit configured to acquire a sound speed of the acoustic matching member and acquire characteristic information of the object based on the sound speed and the electric signal; and

a controlling unit configured to perform control based on information on the temperatures measured at the plurality of positions.

The present invention also provides an information processing method that acquires characteristic information of an object based on an electric signal obtained by receiving, using a conversion element, an acoustic wave that is generated from the object and propagates, the information processing method comprising:

acquiring information on temperatures at a plurality of positions in an acoustic matching member that acoustically matches the object to the conversion element; and

performing control based on the information on the temperatures at the plurality of positions.

According to the present invention, in the apparatus that acquires the object information by using the acoustic wave, it is possible to improve the accuracy of the information acquisition based on the temperature information of the acoustic matching member.

Further features of the present invention will become apparent from the following description of exemplary embodiments with reference to the attached drawings.

### BRIEF DESCRIPTION OF THE DRAWINGS

FIG. 1 is a view showing a configuration of an object information acquiring apparatus of Embodiment 1;

FIG. 2 is a view showing a configuration of an imaging system;

FIG. 3 is a view showing an example of a position of a thermometer disposed in a plane of a bowl-shaped probe;

FIG. 4 is a view showing a configuration of Embodiment to which a thermometer scanning system is added;

FIGS. 5A to 5C are views showing operation flows of an information processing controlling unit of Embodiment 1;

FIG. 6 is a view showing the configuration of the object information acquiring apparatus of Embodiment 2;

FIGS. 7A to 7C are views showing the operation flows of the information processing controlling unit of Embodiment 2;

FIG. 8 is a view showing the configuration of the object information acquiring apparatus of Embodiment 3;

FIGS. 9A to 9C are views showing the operation flows of the information processing controlling unit of Embodiment 3;

FIG. 10 is a view showing the configuration of the object information acquiring apparatus of Embodiment 4;

FIGS. 11A to 11C are views showing the operation flows of the information processing controlling unit of Embodiment 4;

FIG. 12 is a view showing the operation flow of the information processing controlling unit of Embodiment 5; and

FIG. 13 is a view showing a configuration of an apparatus described in Description of the Related Art.

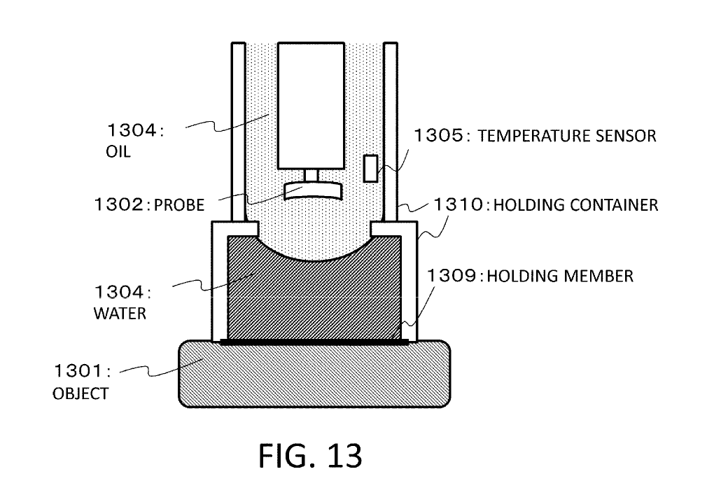

### DESCRIPTION OF THE EMBODIMENTS

Hereinbelow, preferred embodiments of the present invention will be described with reference to the drawings. Note that the dimensions, materials, shapes, and relative dispositions of components described below should be appropriately changed according to the configuration of an apparatus to which the invention is applied and various conditions. Therefore, the scope of the invention is not limited to the following description.

The present invention relates to a technique for detecting an acoustic wave that propagates from an object, and generating and acquiring characteristic information of the inside of the object. Therefore, the present invention is viewed as an object information acquiring apparatus or a control method thereof, or an object information acquiring method or a signal processing method. In addition, the present invention is also viewed as a program that causes an information processing apparatus including hardware resources such as a CPU and a memory to execute the methods, or a storage medium that stores the program.

The object information acquiring apparatus of the present invention includes an apparatus utilizing a photoacoustic effect that receives the acoustic wave generated in the object by irradiating the object with light (electromagnetic wave) and acquires the characteristic information of the object as image data. In this case, the characteristic information is information on characteristic values corresponding to a plurality of positions in the object that are generated by using a reception signal obtained by receiving a photoacoustic wave.

The characteristic information acquired by photoacoustic measurement is a value in which the absorption rate of light energy is reflected. The characteristic information includes, e.g., a generation source of the acoustic wave generated by light irradiation, an initial sound pressure in the object, a light energy absorption density and a light energy absorption coefficient derived from the initial sound pressure, and the concentration of a substance constituting a tissue. It is possible to calculate an oxygen saturation distribution by determining an oxygenated hemoglobin concentration and a reduced hemoglobin concentration as the substance concentration. In addition, a glucose concentration, a collagen concentration, a melanin concentration, and the volume fraction of fat or water are also determined. A two-dimensional or three-dimensional characteristic information distribution is obtained based on the characteristic information at each position in the object. Distribution data can be generated as image data. The characteristic information may be determined not as numerical data but as distribution information at each position in the object. That is, distribution information such as an absorption coefficient distribution or the oxygen saturation distribution may be used as the object information.

The object information acquiring apparatus of the present invention includes an apparatus utilizing an ultrasound echo technique that transmits the ultrasound wave to the object, receives a reflected wave (echo wave) reflected in the object, and acquires the object information as the image data. In the case of the apparatus that utilizes the ultrasound echo technique, the acquired object information is information in which a difference in the acoustic impedance of the tissue in the object is reflected.

The acoustic wave in the present invention is typically the ultrasound wave, and includes an elastic wave called a sound wave or an acoustic wave. An electric signal converted from the acoustic wave by a probe or the like is also referred to as an acoustic signal. Note that the description of the ultrasound wave or the acoustic wave in the present specification is not intended to limit the wavelength of the elastic wave. The acoustic wave generated by the photoacoustic effect is referred to as a photoacoustic wave or an optical ultrasound wave. An electric signal derived from the photoacoustic wave is also referred to as a photoacoustic signal. An electric signal derived from the ultrasound echo is also referred to as an ultrasound signal.

The object information acquiring apparatus in the following embodiments is assumed to be used in diagnosis of a vascular disease of a human or an animal or a follow-up of chemotherapy, for example.

### Embodiment 1

As an example of the object information acquiring apparatus of the present invention, a photoacoustic system that generates the image of the object by applying light to the object and receiving and reconstructing the photoacoustic wave generated from the object will be described.

(Apparatus Configuration)

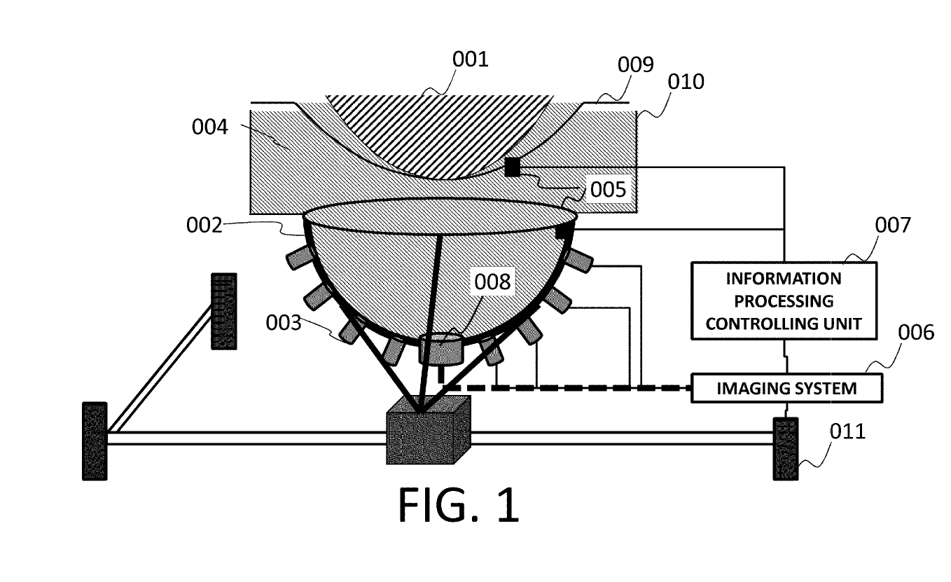

In FIG. 1, an object 001 is a measurement target of which the characteristic information is acquired and the image is generated. A bowl-shaped probe 002 detects the acoustic wave from the inside of the object 001. The probe 002 includes a large number of conversion elements 003. An acoustic matching member 004 is present between the probe 002 and the object 001. A temperature measuring system 005 measures temperatures at a plurality of positions in the acoustic matching member 004.

An imaging system 006 processes an electric signal derived from the acoustic wave received by the group of the conversion elements 003 to reconstruct the image. An information processing controlling unit 007 controls the imaging system 006 based on temperature information acquired by the temperature measuring system 005. A light irradiation unit 008 is an apparatus that applies light to the object 001, and is driven by the imaging system 006. A holding member 009 holds the object 001. A holding container 010 holds the acoustic matching member 004. A driving mechanism 011 changes the relative positional relationship between the probe 002 and the object 001.

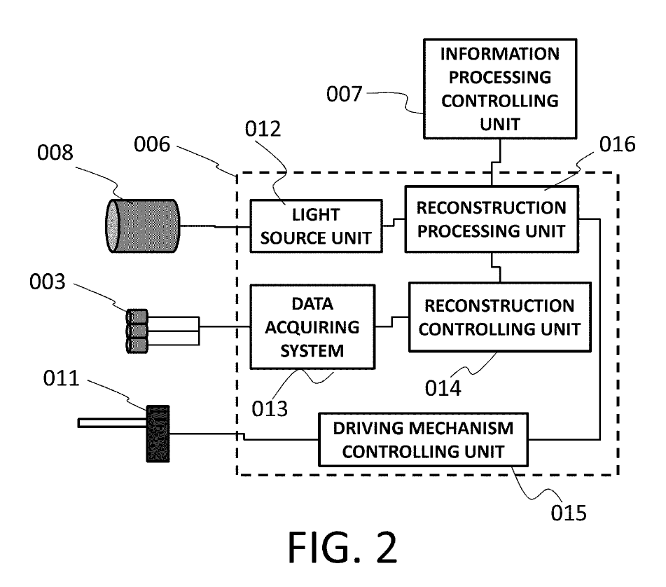

The imaging system 006 is constituted by a plurality of units. The configuration thereof will be described by using FIG. 2. An area corresponding to the imaging system 006 is indicated by a frame in a dotted line. A light source unit 012 is an apparatus in which a light source and a mechanism for controlling the irradiation amount, wavelength, and irradiation timing of the light source are combined. A data acquiring system 013 performs amplification and digital conversion on a signal acquired by the group of the conversion elements 003, and compiles the signal as a data group. A reconstruction processing unit 014 reconstructs the image from the data group output from the data acquiring system 013. A driving mechanism controlling unit 015 controls the operation of the driving mechanism. A reconstruction controlling unit 016 controls the light source unit 012, the reconstruction processing unit 014, and the driving mechanism controlling unit 015.

Note that, in the present embodiment, these units have been described as the imaging system 006 for the sake of convenience. However, when the apparatus is actually configured, the units are not limited to the above classification. It is only necessary for a unit that performs processing described in the following embodiment to exist in the apparatus.

(Photoacoustic Wave Reception and Image Reconstruction by Imaging System)

The imaging system 006 applies light to the object 001 and performs the image reconstruction based on the electric signal derived from the photoacoustic wave generated in the object 001. First, the probe 002 and the light irradiation unit 008 are moved on a two-dimensional plane opposing the object 001 by the driving mechanism 011 controlled by the driving mechanism controlling unit. As the driving mechanism 011, it is possible to use, e.g., a combination of a pulse motor and a ball screw, and a linear motor. The driving mechanism 011 may also be a mechanism that drives the probe 002 in three-dimensional directions. In addition, as the driving mechanism 011, any apparatus may be used as long as the apparatus is capable of position control. Further, one of the object 001 and the probe 002 may be moved or both of them may also be moved.

After the probe 002 and the light irradiation unit 008 reach predetermined positions, light is emitted toward the object 001. The light source of the present embodiment is a Ti: sapphire laser as a type of a solid state laser, and applies pulsed light to the object 001. Its pulse interval is set to 10 Hz. As a laser light source, other than the solid state laser, it is possible to use a gas laser, a dye laser, and a semiconductor laser. In addition, it is also possible to use a flash lamp and a light-emitting diode. As the irradiation light, near infrared rays are preferable. As the wavelength, wavelengths of about 650 to 1100 nm are suitable, and the wavelength is set to 750 nm in the present embodiment. Note that, in order to determine the ingredient concentration and the oxygen saturation of the object 001, it is preferable to use a variable wavelength laser capable of emitting light beams of a plurality of wavelengths. Light is guided from the light source unit 012 to the light irradiation unit 008 by using optical members such as a bundle fiber, a lens, a mirror, and a prism.

When a light absorber in or on the surface of the object 001 absorbs energy of the irradiation light, the acoustic wave is generated due to thermal expansion. Examples of the light absorber that has an absorption characteristic to near-infrared light include blood in a living body that contains a large amount of hemoglobin and melanin. Consequently, a vessel that contains a large amount of blood and a tumor tissue that includes many neovascular vessels easily serve as sound sources, and are preferable as imaging targets.

In the present embodiment, the light irradiation unit 008 is installed at the center of the bowl-shaped probe 002, and moves together with the probe 002. With this, light is efficiently applied to an imaging portion. However, the installation position is not limited to this position. In addition, since the intensity of the photoacoustic wave changes depending on a reached amount of light, even in the case of vessels having the same form, the intensity of the photoacoustic wave differs depending on the depth in the object 001. To cope with this, the imaging system 006 of the present embodiment acquires a light distribution amount in the object 001 by measurement or arithmetic calculation, and uses the light distribution amount in the correction of signal strength. It is preferable to control the irradiation light amount to the object 001 by adjusting light intensity and the position of the light irradiation unit 008.

The acoustic wave generated from the object 001 by the light irradiation passes through the acoustic matching member 004, and is received by the group of the conversion elements 003 of the probe 002. The acoustic matching member 004 acoustically matches the object 001 (or the holding member 009) to the probe 002. Consequently, as the acoustic matching member 004, a member that allows propagation of the acoustic wave and does not prevent scanning of the probe 002 is preferable. Examples of the acoustic matching member include liquids such as water, diisodecyl sebacate(DIDS), polyethylene glycol (PEG), silicone oil, and castor oil. In the present embodiment, water having a surface-active agent is used.

The acoustic wave received by the conversion element 003 is converted to the electric signal in the conversion element 003, and is input to the data acquiring system 013. Amplification, correction, or digital conversion is performed in the data acquiring system 013 on an as needed basis. The data acquiring system 013 can be constituted by an electric circuit or an information processing apparatus that has these functions, or a combination thereof.

The electric signal having been subjected to the processing is input to the reconstruction processing unit 014. In the reconstruction processing unit 014, delay time of a reception signal is determined based on coordinate information of the position of each conversion element 003 in the probe 002, delay processing is performed on each reception signal, and then the image is reconstructed. In each of the image reconstruction processing and the delay time determination, processing in which a digital signal retained in a memory is acquired is performed based on a distance between each conversion element 003 and a target position (a pixel or a voxel) and the sound speed of a propagation substance of the acoustic wave. Accordingly, determination of the sound speed on an acoustic wave path is required in accurate image reconstruction. The sound speed changes according to the temperature and sound usually travels at higher speed as the temperature is higher, and hence it is also important to determine the temperature on the acoustic wave path.

In the reconstruction processing unit 014, it is possible to adopt any known method that uses a band-pass filter or the like. For example, it is possible to use back projection, delay and sum, and Fourier transformation. Note that, when the reconstruction is performed, the image may be reconstructed for each signal acquired at each scanning position of the probe 002, but it is preferable to retain the signals acquired at the individual scanning positions in the memory temporarily and collectively use the signals in the reconstruction. With this, an improvement in SN ratio and an increase in the quality of the image resulting from an increase in the angle of visibility are expected to be achieved.

In the present embodiment, the ratio of the acoustic matching member 004 on the acoustic wave path is large. Accordingly, in order to improve the accuracy of the image reconstruction, the sound speed of the acoustic matching member 004 is acquired based on the temperature of the acoustic matching member 004 acquired by the temperature measuring system 005. An example of the method of acquiring the sound speed from the temperature includes a method in which a table indicative of a correspondence between the temperature and the sound speed is retained for each type of the acoustic matching member in advance and the table is consulted. In addition, it is also possible to calculate the sound speed by reflecting the measured temperature in an expression for calculating the sound speed from the temperature.

The reconstruction processing unit 014 may also be configured by an external PC instead of being configured in the imaging system 006. For example, it is also possible to move the signal output by the data acquiring system 013 to a PC dedicated to the reconstruction and execute the reconstruction offline later. In the present embodiment, the output signal from the data acquiring system 013 can be output to the outside separately such that the reconstruction can be executed online and offline.

The light source unit 012, the reconstruction processing unit 014, and the driving mechanism controlling unit 015 described above are controlled by the reconstruction controlling unit 016. The reconstruction controlling unit 016 determines the start and stop of the reconstruction, the pattern of the imaging, and reconstruction conditions of the image. Each of the reconstruction processing unit 014, the driving mechanism controlling unit 015, and the reconstruction controlling unit 016 can be constituted by an information processing apparatus that includes a CPU and operates with a program.

(Holding Member)

By using the holding member 009, the object 001 is held and the shape thereof is stabilized. With this, the accuracy of the arithmetic calculation such as the arithmetic calculation of an attenuation amount and the calculation of the delay time at the time of the image reconstruction is improved. As the holding member 009, a member having acoustic wave permeability is used. In addition, a material having a small difference in acoustic impedance between the object 001 and the acoustic matching member 004 is preferable. In addition, a member having high stiffness and a member having stretchability are preferable such that the object 001 can be held. Examples of the member having high stiffness include resin materials such as polyethylene terephthalate (PET), polymethyl pentene, and acryl. Examples of the member having stretchability include rubber sheets made of latex and silicone and a material such as urethane. In addition, a holding mechanism obtained by combining a plurality of materials may also be used. In the present embodiment, the cup-shaped holding member 009 formed of a PET material having a thickness of not more than 1 mm is used.

The holding member 009 is preferably installed so as to be replaceable. In the case where a breast is inserted into the apparatus from an opening of a casing, a mounting portion capable of easily fixing the holding member 009 using a metal fitting or a hook is preferably provided around the opening. With this, replacement of the holding member 009 corresponding to a subject and a measurement content is facilitated. In the case where light is emitted from the side of the probe as in the present embodiment, a material having high light permeability is used as the holding member 009.

(Probe)

The conversion element 003 of the probe 002 converts the acoustic wave to the electric signal. As the conversion element 003, it is possible to use lead zirconate titanate (PZT), polyvinylidene fluoride (PVDF), and capacitive micro-machined ultrasonic transducers (cMUT). In addition, it is also possible to use a Fabry-Pérot probe. By using the probe 002 in which a plurality of the conversion elements 003 are disposed on a one-dimensional, two-dimensional, curved, or spherical plane, an improvement in S/N ratio and a reduction in measurement time can be expected to be achieved. In the present embodiment, about 500 circular PZTs each having a diameter of 2 mm are disposed with the center frequency of 2 MHz in the probe 002. The probe 002 has a hemispherical bowl-like shape having a diameter of about 300 mm. The conversion elements 003 are disposed along the inner surface of the hemisphere.

In the present embodiment, the light irradiation unit 008 is installed in the bowl-shaped probe 002, and the probe 002 and the light irradiation unit 008 are simultaneously moved by the driving mechanism 011. However, the light irradiation unit 008 and the probe 002 may also be moved separately. When the bowl-shaped, cup-shaped, or hemispherical probe 002 is used, a high sensitivity area in which directions having high reception sensitivity (directivity axis) of the individual conversion elements 003 are concentrated is formed, and the resolution of the image can be increased. However, the structure of the probe is not limited thereto. For example, a probe having a single element, one-dimensional linear disposition, or two-dimensional planar disposition may also be used.

As the holding container 010, a member having stiffness that can bear water pressure and sealing performance that prevents leakage of liquid to the outside is used. The holding container 010 and the probe 002 can have various positional relationships. For example, the probe 002 may be sunk to a low position in the holding container 010. In this case, the driving mechanism 011 moves the probe 002 in the vicinity of the bottom surface in the holding container 010 such that the probe 002 does not collide with the holding member 009. Such a configuration is especially suitable in the case where a circulation system including the probe 002 and the holding container 010 is provided, as will be described later. In addition, there is also a method in which a member that transmits the acoustic wave and light is disposed on the bottom surface of the holding container, and the probe 002 is moved while the probe 002 is in close contact with the bottom surface. In this case, it is preferable to provide a sealing mechanism or a liquid supplying mechanism such that liquid in the probe 002 is maintained.

(Temperature Measuring System)

The temperature measuring system 005 is an apparatus that has a plurality of thermometers and is for determining a positional variation or difference in the temperature of the acoustic matching member 004 (temperature distribution). This temperature information is used in equalization of the temperature distribution and the image reconstruction in which the actual situation of the temperature distribution is reflected. Particularly in the case where the object 001 is a living body such as the breast, the breast often serves as a heat source. Accordingly, there is a high possibility that a difference in temperature between the vicinity of the object 001 and the vicinity of the probe 002 occurs in the acoustic matching member 004. To cope with this, in the present invention, temperatures at a plurality of positions in the acoustic matching member 004 are measured and the temperature distribution is obtained. Subsequently, it becomes possible to calculate a sound speed distribution situation from the temperature distribution, and reflect the sound speed distribution situation in the reconstruction condition or use the sound speed distribution situation in temperature equalization. The temperature measuring system corresponds to a temperature measuring unit of the present invention.

In the temperature measurement, a resistance thermometer such as a thermo-electric pile or a thermistor is suitable. In addition, the use of a thermometer capable of collectively measuring the wide-range temperature distribution in a noncontact manner such as a thermography is also effective. However, since the thermography is a system that measures the temperature of the water surface or the temperatures of wall surfaces of the holding member 009 and the holding container 010 through water, setting of the installation position and the measurement position requires thoughtful devising. That is, the measurement position is set such that the propagation path of the sound wave described later can be covered.

As the measurement position, the position that allows ascertainment of the temperature on the propagation path of the reception signal such as the position in the vicinity of the conversion element 003 constituting the probe 002 or the position in the vicinity of the object 001 that is the imaging target and serves as the heat source is preferable. Note that, while the present embodiment describes a photoacoustic system, it is preferable that the temperature of the acoustic matching member 004, present between the conversion element 003 that applies the ultrasound wave and the object 001 as the imaging target, can also be ascertained in an imaging system using an ultrasound echo.

In order to ascertain the temperature in the peripheral portion of the acoustic matching member 004, the temperature of a member in contact with the acoustic matching member 004 may also be measured instead of the acoustic matching member 004 itself. The member in contact therewith is the holding member 009, the probe 002, the conversion element 003, or the holding container 010. In the case where the holding member 009 is configured to conduct heat efficiently, the surface temperature of the object 001 serving as the heat source may be measured. However, in the case where the temperature of the acoustic matching member 004 itself is not measured, it is necessary to determine the relationship between the measured temperature and the temperature of the acoustic matching member 004 in advance. As the method of determining the relationship therebetween, it is possible to use the result of an experiment or a simulation.

The accuracy of the calculation of the sound speed distribution is improved by increasing the number of installation positions of the thermometer, but the increase in the number of installation positions leads to degradation of the reconstructed image because the thermometer interrupts the sound path. Consequently, it is preferable to determine the number of installed thermometers and the installation position while striking a balance between temperature distribution acquisition accuracy and propagation path securement. In the present embodiment, small thermistors are installed at several positions (e.g., three to four positions) on the surface of the holding member 009 on the side of the acoustic matching member 004 and on the surface of the bowl-shaped probe 002.

By ascertaining the temperature change and distribution in advance by the experiment or the simulation, it becomes possible to reduce the number of temperature measurement positions and perform the measurement at a position that does not interrupt the sound path. In addition, it is also preferable to appropriately adjust the measurement position in accordance with the configuration of the object information acquiring apparatus. When a factor for the temperature variation is present such as, e.g., the case where the acoustic matching member is circulated or the case where a liquid temperature adjusting apparatus is provided for improving comfortability of the subject, the measurement position corresponding to the factor may be appropriately set.

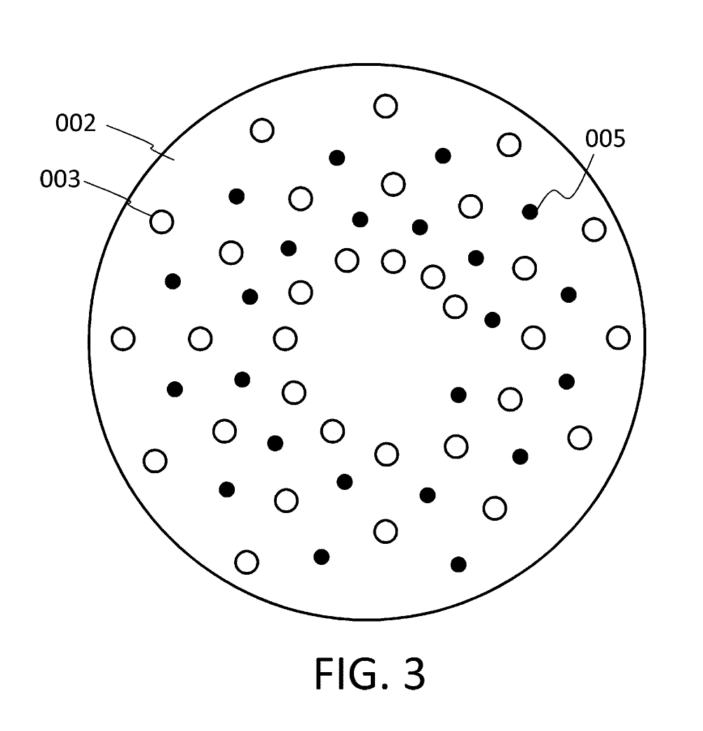

In addition, as shown in FIG. 3, there is a method in which the thermometer of the temperature measuring system 005 is disposed between the conversion elements 003 in the probe 002. Further, there is a method in which the thermometer is disposed in the conversion element 003. According to these methods, it is possible to ascertain the temperature at the position of each conversion element 003. In addition, it is also possible to ascertain the temperature distribution on the sound path of the reception signal with high accuracy by disposing a plurality of temperature sensors in the holding member 009.

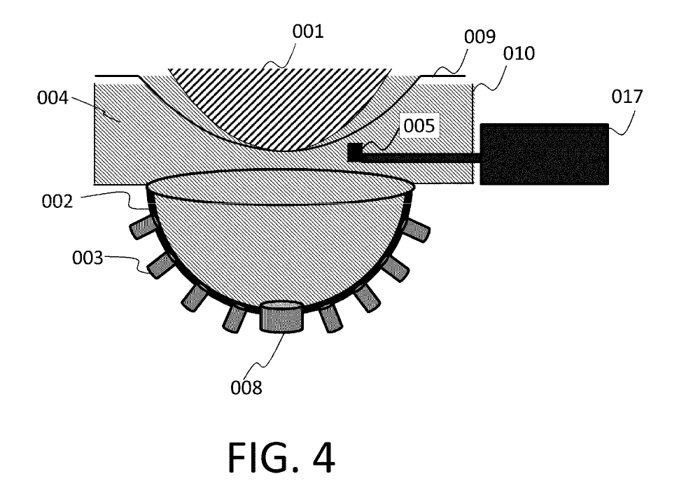

In addition, as shown in FIG. 4, it is also preferable to install a thermometer scanning system 017 capable of moving the installation position of the thermometer to any position in the acoustic matching member 004. With this, it is possible to freely measure the temperature in the acoustic matching member 004. The thermometer scanning system 017 may perform pre-programmed operations, or the installation position may also be moved manually by an operator. FIG. 4 shows one thermometer. However, a plurality of the thermometers may also be provided.

(Operation Example of Information Processing Controlling Unit)

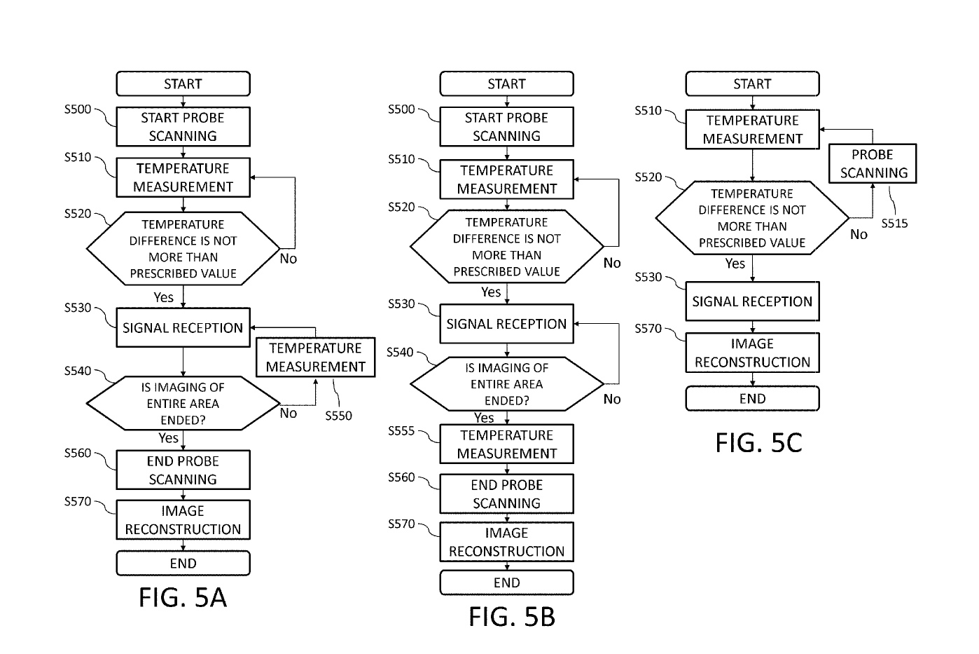

The operation of the information processing controlling unit 007 in the present embodiment will be described by using FIG. 5. In FIG. 5A, when the system is started, the probe 002 and the holding container 010 are caused to start scanning by the driving mechanism 011 (Step S500). Then, each temperature information measured in the temperature measuring system 005 is transmitted to the information processing controlling unit 007 (Step S510). Subsequently, information indicative of the temperature distribution in the situation in which the object 001 is disposed in the holding member 009 that is used in the reconstruction is acquired. In the present embodiment, as the information indicative of the temperature distribution, difference information of the temperature of the acoustic matching member 004 measured in each thermometer is determined. In the case where there are three or more thermometers, the difference information is determined by comparing the maximum value with the minimum value.

Then, the information processing controlling unit 007 determines whether or not the difference information of the temperature is not more than a predetermined prescribed value. When the difference information is not more than the prescribed value, the information processing controlling unit 007 issues an instruction to perform the imaging to the reconstruction controlling unit 016 (Step S520). This is because it is possible to assume that the temperature variation on the acoustic wave propagation path in the acoustic matching member 004 is small in the case where the temperature difference is small. In the present embodiment, the predetermined prescribed value is set to 0.5° C. Note that the prescribed value can be appropriately changed according to the configuration of the apparatus and requested image quality. For example, in the case where the distance within the acoustic matching member 004 between the probe 002 and the object 001 is small or in the case where requested resolution is low, the prescribed value can be set to be high.

Subsequently, signal reception is performed (Step S530). That is, light irradiation and photoacoustic wave reception are performed in the case of photoacoustic measurement, and ultrasound wave transmission and echo wave reception are performed in the case of ultrasound echo measurement. When the reconstruction is executed based on the signal received in this state, it is possible to assume that the sound speed of the acoustic matching member 004 is constant. An inputting unit constituted by a user interface such as a keyboard, a mouse, or a touch panel for a user to directly input the prescribed value or input the requested image quality may also be provided.

In the case where the difference information is more than the prescribed value, the imaging is not executed and the scanning of the probe 002 is continued. The acoustic matching member 004 is stirred by the scanning of the probe 002, and hence the temperature variation is gradually reduced. Subsequently, when the difference information calculated in the temperature measuring system 005 becomes equal to or less than the prescribed value, the imaging is started.

The measurement of reception data and temperature data at each coordinate is continued until all measurement areas are imaged (Step S540). On the other hand, when the imaging of all of the measurement areas is not completed, the temperature measurement and the signal reception at another position are performed again (Step S550). At this point, reduction processing of the temperature variation may be performed in accordance with the result of the temperature measurement in S550. Subsequently, after the completion of the imaging of all of the measurement areas, the scanning of the probe 002 is stopped (Step S560). Lastly, the reconstruction is executed based on the acquired imaging data (Step S570). In the present embodiment, the temperature is measured at each signal reception position (S550). The reconstruction processing unit 014 does not use the reception data at the position where the difference information is more than the prescribed value in the reconstruction. With this, characteristic information having high accuracy is acquired. Note that, in the case where the reduction processing of the temperature variation is performed at each signal reception position until the difference information based on the result of the temperature measurement in Step S550 becomes equal to or less than the prescribed value, data suitable for the reconstruction is obtained over the entire area, and hence the accuracy of the characteristic information is further improved.

(Another Operation Example of Information Processing Controlling Unit)

Subsequently, the description will be made with reference to FIG. 5B. This shows the case where the temperature is measured only when the imaging is started or only when the imaging is started and ended, the temperature difference information is predicted during the measurement time without performing the measurement, and the temperature difference information is consulted when the reconstruction is performed. This method is effective especially in the case where the temperature change at the time of the measurement is small due to the heat capacity of the acoustic matching member 004 and material characteristics of the surface of the probe 002, the holding member 009, and the holding container 010. This method is also effective in the case where the distance within the acoustic matching member 004 between the conversion element 003 and the object 001 is short.

In FIG. 5B, the temperature is measured at the time of the start (Step S510) and at the time of the end (Step S555). The timing of the measurement is preferably set at the start and end of the reception of the acoustic wave. At the time of the start, the scanning of the probe 002 is continued until the difference information becomes equal to or less than the prescribed value (S510 and S520). After the difference becomes equal to or less than the prescribed value and the imaging is started, the measurement of the temperature is not executed until the imaging is ended. Subsequently, after the end of the imaging, the temperature measurement is performed in Step S555. When the image reconstruction is performed, estimation processing of the temperature distribution and the sound speed distribution at the point of time of the imaging at each position is performed based on the first and last difference information items (or the temperature distribution information itself). In the reconstruction of the present flow, it is preferable to use the result of the experiment or the simulation executed in advance.

In this method, since the temperature measurement in the middle of the operation is not necessary, it becomes possible to perform the temperature measurement at the position that interrupts the reconstruction such as the position on the sound path. Consequently, by using the thermometer scanning system 017 in FIG. 4, it is possible to set the thermometer on the sound path before the imaging is started and after the imaging is ended, and retract the thermometer from the position on the sound path during the imaging.

(Another Operation Example of Information Processing Controlling Unit)

It is also possible to cause the reconstruction controlling unit 016 to operate such that the reconstruction method for the reception signal in the case where the temperature difference is not less than the prescribed value is different from the reconstruction method for the reception signal in the case where the temperature difference is not more than the prescribed value. For example, when the operation is executed according to the flow in FIG. 5A, the image reconstruction is performed by using an equalized sound speed in which the sound speed of the acoustic matching member 004 is considered constant in the case of the signal received when the temperature difference is not more than the prescribed value. On the other hand, in the case of the signal received when the temperature difference is not less than the prescribed value, a plurality of sound speeds in the acoustic matching member 004 are set, and the image is reconstructed for each sound speed. The optimum sound speed can be calculated by comparing and evaluating the reconstructed images. With this, it is possible to reduce the processing amount to a level lower than that in the case where the condition of the sound speed is set in each of all signals used in the reconstruction.

Note that, as a reference sound speed when the equalized sound speed or the optimum sound speed is calculated, it is possible to consult the sound speed based on the average of values acquired with a plurality of the thermometers and the sound speed based on the acquired value in any of the thermometers. In the present embodiment, the temperature on the side of the probe 002 is consulted. This is because, in the use in the present embodiment, the temperature tends to change sharply in the vicinity of the holding member 009 in the temperature change between the probe 002 and the holding member 009, and the sound speed on many sound paths tends to be based on the temperature on the side of the probe 002. However, the tendency is not limited thereto actually.

In the configuration in FIG. 3, the temperature of each conversion element 003 at the time of the reconstruction is measured by installing the thermometer between the conversion elements 003 of the probe 002 or in the conversion element 003. In this case, it is possible to ascertain the temperature difference on the sound path of the reception signal for each conversion element 003 at each reconstruction position. Accordingly, among the reception signals, only the reception signal with which the sound speed can be assumed to be constant can be used in the reconstruction.

(Another Operation Example of Information Processing Controlling Unit)

In the case where data of only one shot is acquired, it is possible to adopt the flow in FIG. 5C. In this case, after the start, the temperature difference is calculated after the probe 002 is moved to the measurement position, and the temperature difference is compared with the prescribed value. When the temperature difference is more than the prescribed value, the probe 002 is moved and the acoustic matching member 004 is stirred (Step S515). Subsequently, the probe 002 is moved to the measurement position again, and the temperature difference is calculated. This operation is continued until the temperature difference becomes equal to or less than the prescribed value, and the imaging is executed.

Thus, in the present invention, the temperatures at a plurality of positions in the acoustic matching member 004 present between the object 001 and the probe 002 are measured, the difference therebetween is determined, and the difference is reflected in the reconstruction. As a result, it is possible to determine whether or not the sound speed in the acoustic matching member 004 can be assumed to be constant in the calculation of the delay time in the processing of the reception signal at the time of the reconstruction, and it is possible to realize simplification of the reconstruction and high image quality of the reconstructed image. Consequently, in the object information acquiring apparatus that uses the photoacoustic wave or the ultrasound echo, it is possible to improve the accuracy of information acquisition based on the temperature information of the acoustic matching member.

### Embodiment 2

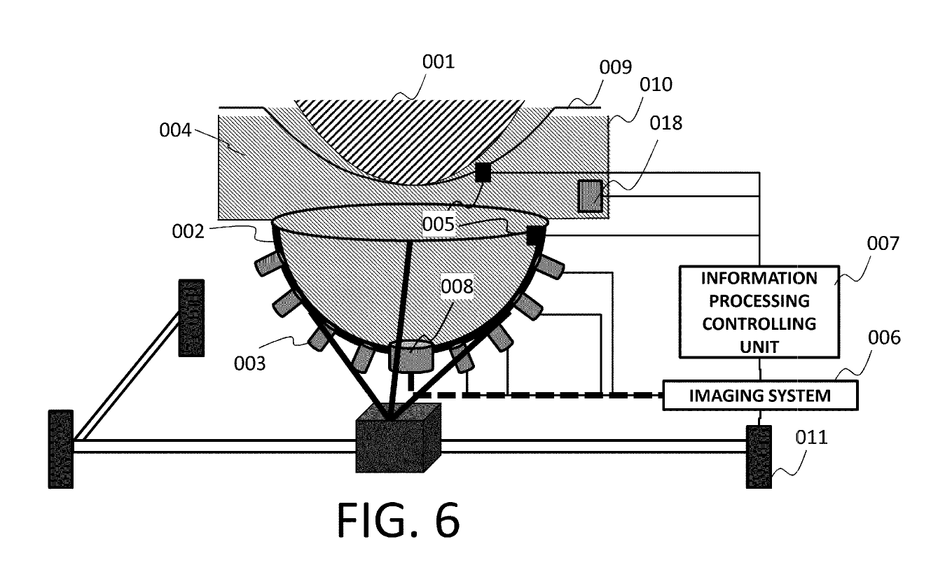

FIG. 6 shows a system schematic diagram of the object information acquiring apparatus of the present embodiment. The same components as those in the above-described embodiment are designated by the same reference numerals, and the detailed description thereof will be omitted. In the present embodiment, a temperature adjuster 018 that adjusts the temperature of the acoustic matching member 004 is additionally provided.

In the present embodiment, water having a surface-active agent is used as the acoustic matching member 004, and the water temperature tends to be stabilized in the vicinity of the temperature of a room in which the object information acquiring apparatus is placed. Accordingly, in the present embodiment, the water temperature is set to about 20° C. to 30° C. In addition, when the object 001 is disposed in the apparatus, the water temperature in the vicinity of the object 001 is influenced by the temperature of the object 001. In the present embodiment, the breast is assumed as the object 001, and the surrounding water temperature is likely to rise due to the influence of body temperature. Further, for the comfortability of the subject, there are cases where hot water is put in the peripheral portion of the breast. In these cases, the water temperature in the vicinity of the object 001 is 35° C. to 40° C. The temperature adjuster 018 is installed in order to adjust the water temperature by heating the water as the acoustic matching member 004 such that the water temperature corresponds to the body temperature.

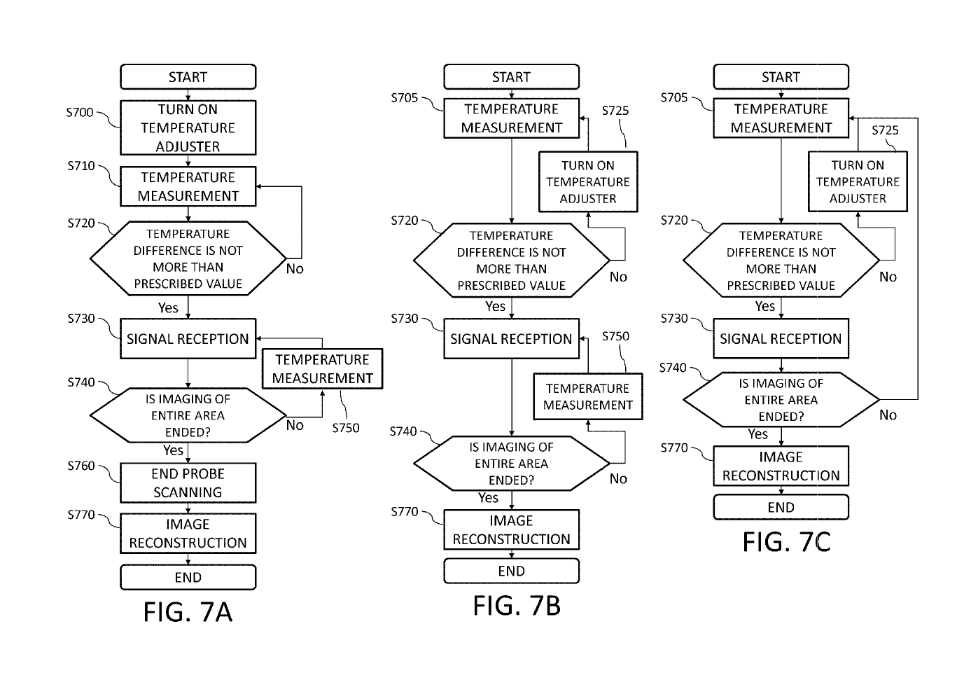

First, as in the flow in FIG. 7A, before photographing, the temperature adjuster 018 is activated in advance (Step S700). Subsequently, at the time of start of the photographing, the individual temperature information items measured in the temperature measuring system 005 are compared, and the difference information is acquired and compared with the prescribed value (Steps S710 and S720). In the case where the difference information is not more than the prescribed value, the imaging is started. Thereafter, with the same processing as that in Steps S530 to S570 in FIG. 5A, image data is obtained (Steps S730 to S770).

In addition, as in the flow in FIG. 7B, there are cases where the temperature adjuster 018 is not activated immediately after the processing start. That is, the difference information at the time of start of the photographing is compared with the prescribed value first (Step S720). When the difference is not more than the prescribed value, the temperature adjuster 018 is automatically operated (Step S725). As soon as the difference becomes equal to or less than the prescribed value, the imaging is started.

In addition, as in the flow described in FIG. 7C, it is also effective to reduce power consumption by constantly monitoring the difference and turning ON/OFF the drive of the temperature adjuster 018 with the prescribed value used as the reference of the switching. In the present flow, when the temperature difference is not more than the prescribed value at a given position, the temperature adjuster 018 is turned OFF (Step S727). Subsequently, the temperature measurement is performed at another position after movement by the scanning again (Step S705).

A target temperature of the temperature adjuster 018 may be input by assuming the temperature of the object 001 in advance by the operator, or may also be determined based on the temperature acquired in the temperature measuring system 005. It is preferable to set the target temperature to the temperature (typically not less than 35° C. and not more than 40° C.) in the vicinity of the object 001 that can be the heat source in terms of the comfortability of the subject and in a point that the temperature can be adjusted to the target temperature in a short time period due to a small adjustment amount.

The photographing of the breast was executed by using the system described above. According to the present system, it became possible to perform control such that the temperature distribution of the acoustic matching member 004 is diminished by using the temperature adjuster 018. As a result, it became possible to smoothly start the imaging. In addition, it became possible to maintain the temperature in the vicinity of the breast to the vicinity of the body temperature, and it was possible to reduce the burden of the subject.

### Embodiment 3

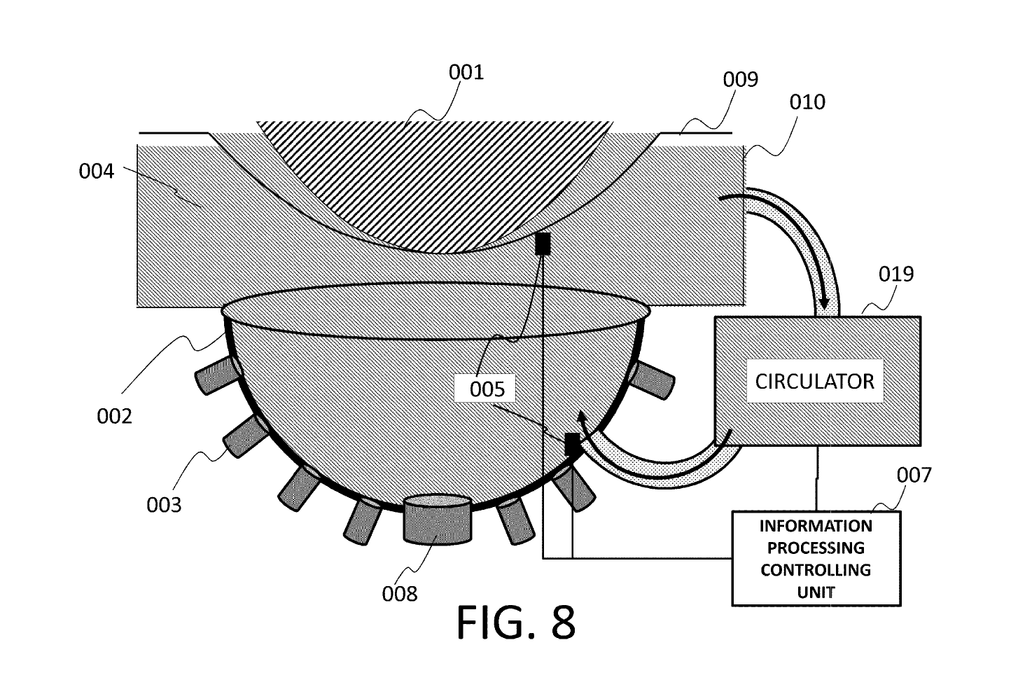

FIG. 8 shows a system schematic diagram of the object information acquiring apparatus of the present embodiment. The same components as those in the above-described embodiments are designated by the same reference numerals, and the detailed description thereof will be omitted. In the apparatus of the present embodiment, a circulator 019 that circulates the acoustic matching member 004 in the holding container 010 and the bowl-shaped probe 002 is additionally provided. The circulator 019 circulates the acoustic matching member 004, and the temperature variation of the acoustic matching member 004 is thereby reduced.

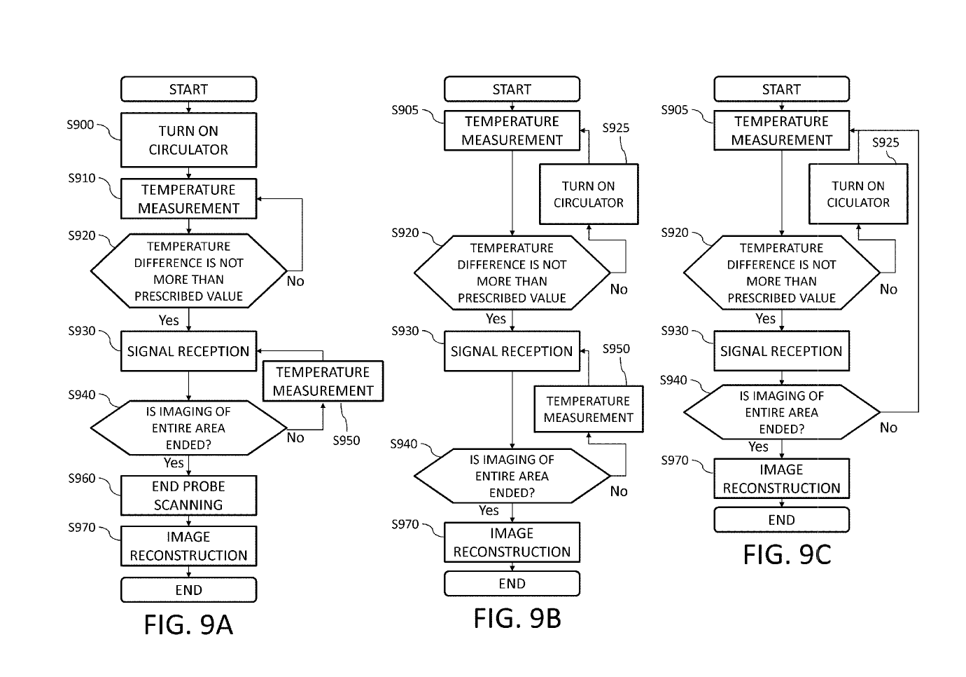

In the flow in FIG. 9A, first, the circulator 019 is activated in advance before the photographing (Step S900). Subsequently, the difference between the temperature measured in the temperature measuring system 005 at the time of start of the photographing and the prescribed temperature is verified. In the case where the difference is not more than the prescribed value, the imaging is started (Steps S910 to S930). Thereafter, the scanning is performed and the characteristic information of the wide area of the object is obtained (Steps S940 to S970). In the case where it is determined that the desired temperature distribution is not obtained in the temperature measurement (Step S950) in the middle of the flow, the object information acquiring apparatus may wait for optimization of the temperature by liquid circulation by the circulator or stirring by the probe. On the other hand, in the case where the temperature difference is more than the prescribed value, the processing such as the reception of the acoustic wave or the light irradiation in the photoacoustic measurement is not started.

In the flow in FIG. 9B, the circulator 019 is not activated in advance. In this case, the temperature at the time of start of the photographing is measured, and the difference comparison is performed (Steps S905 and S920). Subsequently, when the difference is not more than the prescribed value, the circulator 019 is automatically operated (Step S925). Then, as soon as the temperature difference becomes equal to or less than the prescribed value, the imaging is started (Steps S930 to S970).

In the flow in FIG. 9C, the temperature difference is constantly measured during the scanning. The drive of the circulator 019 is turned ON/OFF with the prescribed value used as the reference of the switching (Steps S925 and S927). This is effective at reducing power consumption.

The outlet and the inlet of the circulator 019 are preferably disposed in accordance with the configuration of the imaging apparatus of the object 001. In the present embodiment, as indicated by arrows in FIG. 8, the outlet is provided in the probe 002, and the inlet is provided in the holding container 010. In this case, liquid moves from the probe 002 toward the holding member 009. As a result, the temperature on the sound path from the conversion element 003 to the object 001 is influenced by the temperature of the circulated liquid. In addition, the liquid in the vicinity of the holding member 009 influenced by the temperature of the object 001 is replaced with the circulated liquid. As a result, it is possible to efficiently equalize the temperature distribution.

In the configuration in FIG. 8, it is preferable to install the thermometer constituting the temperature measuring system 005 in each of the outlet portion of the circulator 019 and the holding member 009. By controlling the flow rate and direction of the liquid that is pushed and flown toward the object 001 from the vicinity of the probe 002 in accordance with the measurement result, it is possible to execute the temperature adjustment accurately and easily. The positions of the outlet and the inlet in FIG. 8 may be reversed. In addition, it is also preferable to allow switching between the outlet and the inlet. Further, in a configuration in which the holding container 010 and the probe 002 are separated from each other, the circulator may be provided in the holding container 010, the probe 002, or each of the holding container 010 and the probe 002.

There are cases where the circulator 019 discharges bubbles during the circulation of water depending on the type of the circulator 019. The adhesion of the bubbles to the holding member 009 causes degradation of the image, and hence it is not preferable to drive the circulator 019 when the outlet of the circulator 019 is positioned immediately below the holding member 009. In this case, it is preferable to drive the circulator 019 in a situation in which the outlet is moved away from the position immediately below the holding member 009 by using the driving mechanism 011.

The photographing of the breast was executed by using the system described above. It became possible to perform control such that the temperature distribution of the acoustic matching member 004 was diminished by using the circulator 019 and smoothly start the imaging, and the image having high resolution was obtained.

### Embodiment 4

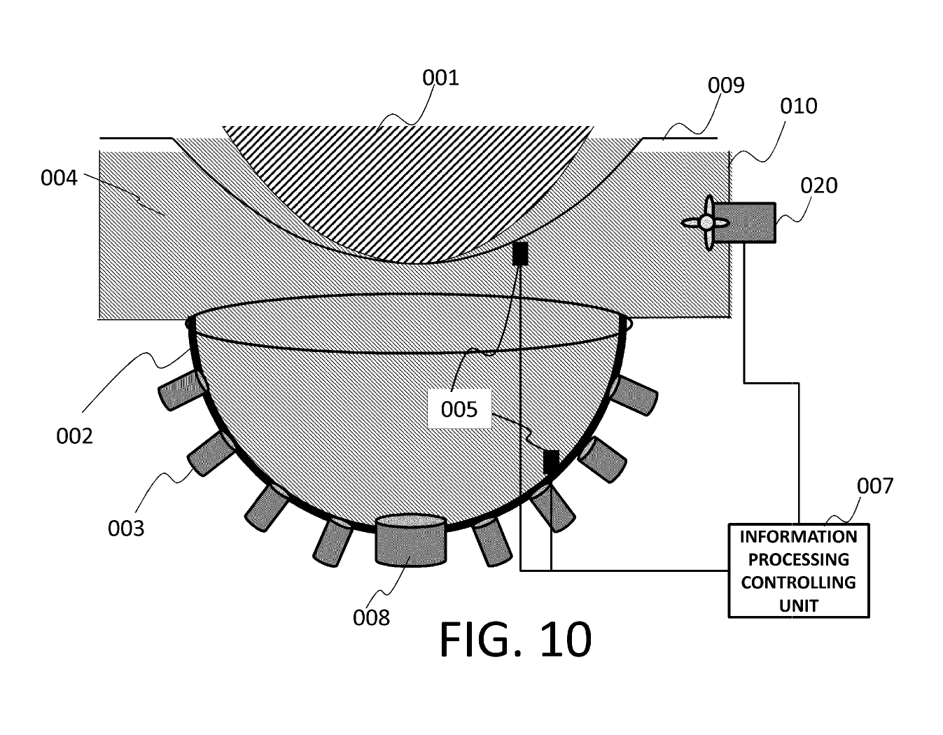

FIG. 10 shows a system schematic diagram of the object information acquiring apparatus of the present embodiment. The same components as those in the above-described embodiments are designated by the same reference numerals, and the detailed description thereof will be omitted. The apparatus of the present embodiment has a stirrer 020 that stirs the acoustic matching member 004 in the holding container 010 and the probe 002. As described in each embodiment described above, the temperature variation tends to occur in the acoustic matching member 004 due to influences of the body temperature and an operation heat of the apparatus. To cope with this, in the present embodiment, the variation (unevenness) in the temperature distribution is reduced by stirring the acoustic matching member 004 such as water by using the stirrer 020.

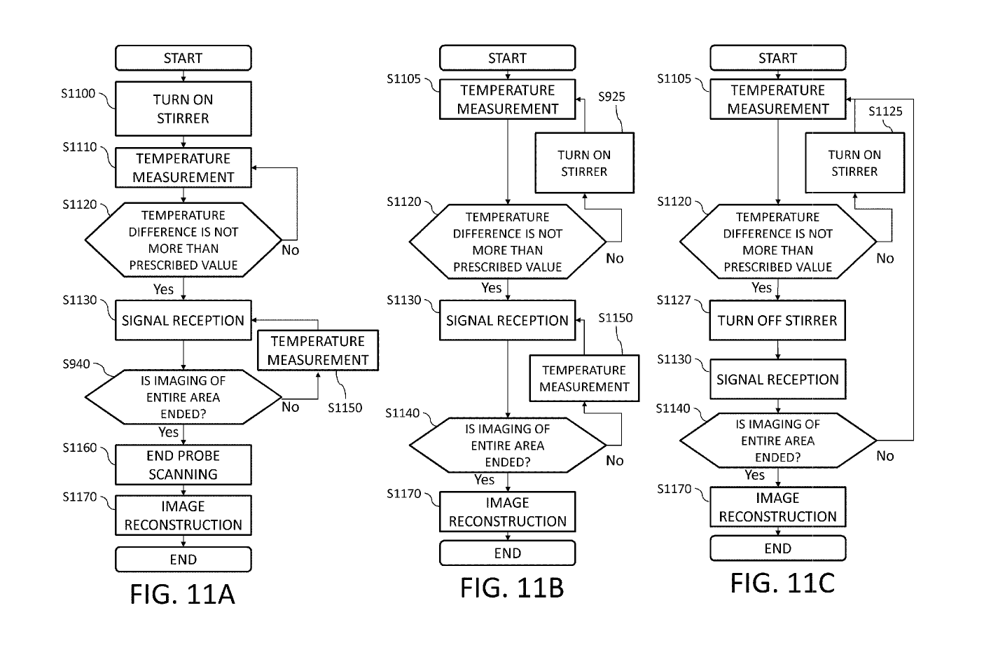

In the flow in FIG. 11A, before the photographing, the stirrer 020 is activated in advance (Step S1100). Subsequently, the temperature is measured in the temperature measuring system 005 at the time of start of the photographing, the comparison processing is performed, and the difference information is verified (Steps S1110 to S1120). In the case where the comparison result is not more than the prescribed value, the imaging is started and the characteristic information is acquired (Steps S1130 to S1170). In the case where the temperature variation is detected in the temperature measurement in the middle of the flow (Step S1150), the stirring processing may be performed.

In the flow in FIG. 11B, the stirrer 020 is not activated in advance. When the temperature difference at the time of start of the photographing is not more than the prescribed value, the stirrer 020 is automatically operated (Steps S1105, S1120, and S1125). With this, it is possible to start the imaging in a state in which the temperature difference is not more than the prescribed value.

In the flow in FIG. 11C, the temperature difference is constantly measured, and the drive of the stirrer 020 is turned ON/OFF with the prescribed value used as the reference of the switching (Steps S1127 and S1125). With this, there is achieved an effect of reducing power consumption.

In an example in FIG. 10, a mechanism that rotates a screw is adopted as the stirrer 020. At this point, it is preferable to flow the stream of water toward the holding member 009. As a result, water in the vicinity of the holding member 009 influenced by the temperature of the object is replaced, and hence the temperature variation is efficiently reduced.

The photographing of the breast was executed by using the system described above. It became possible to perform control such that the temperature distribution of the acoustic matching member 004 is diminished by using the stirrer 020 and smoothly start the imaging, and the image having high resolution was obtained.

### Embodiment 5

A system schematic diagram of the object information acquiring apparatus of the present embodiment is assumed to include all of the contents shown in FIGS. 1, 6, 8, and 10. The same components as those in the above-described embodiments are designated by the same reference numerals, and the detailed description thereof will be omitted. The apparatus of the present embodiment performs estimation processing of the sound speed distribution by using the temperature difference information, and reflects the processing result in the reconstruction. Specifically, the apparatus has a configuration in which the equalized sound speed is adopted when the difference is not more than the prescribed value at the time of the temperature measurement, and the sound speed distribution in the acoustic matching member is estimated in the case where the difference is not less than the prescribed value and the estimation result is reflected in the reconstruction.

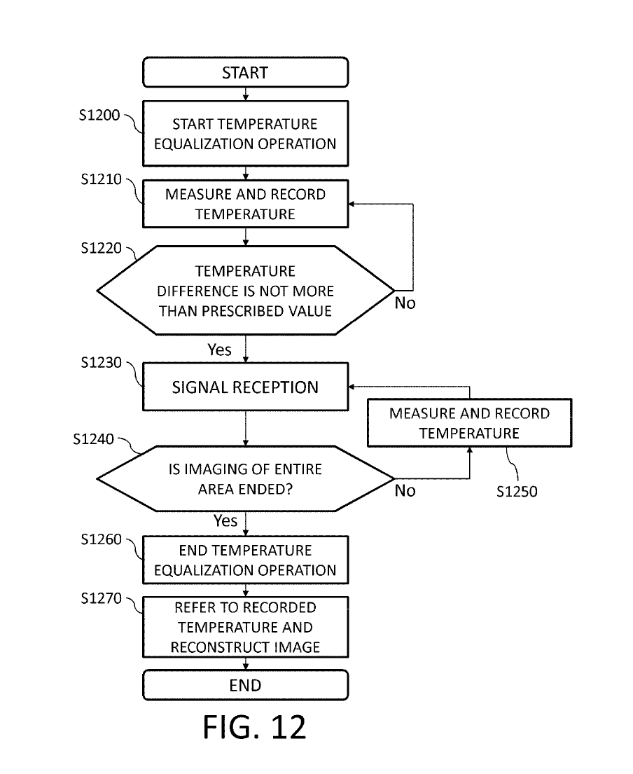

As shown in the flow in FIG. 12, first, before the photographing, a temperature equalization operation is started in advance (Step S1200). The temperature equalization operation means a method such as the scanning of the probe 002, the circulation of the liquid by the circulator 019, the stirring by means of the stirrer 020, or the temperature adjustment by means of the temperature adjuster 018. A plurality of the methods may be combined. Thereafter, similarly to the other embodiments, the temperature difference information is verified in the temperature measuring system 005 at the time of start of the photographing, and the imaging is started in the case where the temperature difference is not more than the prescribed value (Steps S1210 to S1230). When the imaging of the entire area is ended, the temperature equalization operation is ended (Steps S1240 and S1260). Herein, in the case where it is determined that the desired temperature distribution is not obtained in the temperature measurement in the middle of the flow (Step S1250), the object information acquiring apparatus may wait until the temperature equalization operation takes effect. Herein, in Steps S1210 and S1250, the measured temperature is recorded in a recording apparatus of the information processing apparatus, such as a memory. Subsequently, the temperature information is used in the image reconstruction (Step S1270).

In addition, in the present embodiment, instead of executing the temperature equalization operation in advance as in FIGS. 7B, 9B, and 11B, it is also possible to automatically start the temperature equalization operation when the temperature difference at the time of start of the photographing is not more than the prescribed value. Further, in the present embodiment, as shown in FIGS. 7C, 9C, and 11C, it is also effective to reduce power consumption by measuring the temperature difference and turning ON/OFF the execution of the temperature equalization operation with the prescribed value used as the reference of the switching. In the case where data of only one shot is acquired, the flow similar to that in FIG. 5C may be used.

In the present embodiment, for each reception signal at each scanning position, the difference information of the temperature at the time of the imaging is measured or predicted. When the difference information is not more than the prescribed value, similarly to the other embodiments, the equalized sound speed is adopted. On the other hand, in the case where the temperature difference is not less than the prescribed value, the distribution of the sound speed is estimated, and the estimation result is reflected in the reconstruction. Note that the prescribed value in the present embodiment is set to 0.5° C.

The estimation of the distribution of the sound speed can be performed based on a simulation that uses a finite element method or the like or a measured value obtained by an experiment. In the present embodiment, the temperature distribution of each temperature equalization operation in the present system is measured and detabased in advance, and the temperature distribution can be predicted from the temperature difference that can be measured in the temperature measuring system 005.

However, since the calculation amount of the sound speed distribution estimation is large, it sometimes takes time. To cope with this, in a preview of the reconstructed image immediately after the imaging, the image reconstructed only with the reception data when the temperature difference is not more than the prescribed value may be used. With this, it becomes possible to perform real-time or almost real-time display. On the other hand, in the case where the reconstruction is performed offline later, when the reconstruction is performed by using data obtained by executing the sound speed distribution estimation, it is possible to perform imaging with high accuracy.

In the estimation of the sound speed distribution, when the temperature distribution in the acoustic matching member is small, the accuracy of the estimated sound speed is improved. Accordingly, also in the method of estimating the sound speed distribution, it is preferable to equalize the temperature. To cope with this, it is effective to have the prescribed value of the difference information for determining the imaging start (first prescribed value) and the prescribed value of the difference information for prescribing the execution of the sound speed distribution estimation (second prescribed value) separately. For example, in the present embodiment, the prescribed value for determining the imaging start in Step S1220 is set to 1.0° C. With this, it is possible to prevent an increase in waiting time for equalizing the temperature while reducing the temperature variation to a predetermined level or lower. The prescribed value for determining whether or not the estimation of the sound speed in Step S1270 is executed is set to 0.5° C. With this, the high-resolution image in which the sound speed is accurately reflected is obtained. The reconstruction processing unit 014 uses the equalized sound speed when the difference information is not more than the prescribed value (0.5° C.), and uses the estimated sound speed when the difference information is more than the prescribed value. According to this processing, it is possible to acquire the characteristic information with high accuracy while suppressing the use amount of an information processing resource.

The photographing of the breast was executed by using the system described above. It becomes possible to efficiently acquire the image having high image quality by using the equalized sound speed and the estimated sound speed differently.

Thus, according to each embodiment of the present invention, in the apparatus that acquires the object information by using the ultrasound wave, it is possible to improve the accuracy of the information acquisition based on the temperature information of the acoustic matching member. Particularly in the object information acquiring apparatus in which the acoustic matching member is present between the object and the probe, it is possible to ascertain the temperature distribution in the acoustic matching member from the temperature difference information at a plurality of positions in the acoustic matching member. In addition, by executing the reconstruction in the situation in which the sound speed can be assumed to be constant, it is possible to realize simplification of the reconstruction and high image quality of the reconstructed image.

### OTHER EMBODIMENTS

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-199260, filed on Oct. 7, 2015, which is hereby incorporated by reference herein in its entirety.

What is claimed is:

1. An object information acquiring apparatus comprising:
a conversion element configured to receive an acoustic wave that is generated in and propagated from an object and convert the acoustic wave to an electric signal;
a holding unit configured to hold an acoustic matching member that acoustically matches the object to the conversion element;
a temperature measuring unit configured to measure temperatures at a plurality of positions in the acoustic matching member;
a reconstruction processing unit configured to acquire a sound speed of the acoustic matching member and acquire characteristic information of the object based on the sound speed and the electric signal; and
a controlling unit configured to perform control based on information on the temperatures measured at the plurality of positions.

2. The object information acquiring apparatus according to claim 1, wherein
the controlling unit is configured to perform the control based on difference information of the temperatures measured at the plurality of positions.

3. The object information acquiring apparatus according to claim 1, further comprising:
a driving mechanism configured to change a relative positional relationship between the conversion element and the object, wherein
the controlling unit is configured to stir the acoustic matching member by controlling the driving mechanism based on the information on the temperatures measured at the plurality of positions.

4. The object information acquiring apparatus according to claim 3, wherein
the controlling unit is configured to stir the acoustic matching member by controlling the driving mechanism in a case where the difference between the temperatures measured at the plurality of positions is more than a first prescribed value.

5. The object information acquiring apparatus according to claim 1, further comprising:
a temperature adjuster configured to change each of the temperatures of the acoustic matching member, wherein
based on the information on the temperatures measured at the plurality of positions, the controlling unit controls the temperature adjuster such that a difference between the temperatures is reduced.

6. The object information acquiring apparatus according to claim 5, wherein
the controlling unit is configured to control the temperature adjuster such that the difference between the temperatures is reduced in a case where the difference between the temperatures measured at the plurality of positions is more than a first prescribed value, and after controlling the temperature adjuster such that the difference between the temperatures is reduced, control the reconstruction processing unit such that the reconstruction processing unit acquires the sound speed.

7. The object information acquiring apparatus according to claim 1, further comprising:
a circulator configured to circulate the acoustic matching member, wherein
based on the information on the temperatures measured at the plurality of positions, the controlling unit controls the circulator such that a difference between the temperatures is reduced, and after controlling the circulator such that the difference between the temperatures is reduced, controls the reconstruction processing unit such that the reconstruction processing unit acquires the sound speed.

8. The object information acquiring apparatus according to claim 1, further comprising:
a stirrer configured to stir the acoustic matching member, wherein
the controlling unit is configured to control the stirrer such that a difference between the temperatures is reduced based on the information on the temperatures measured at the plurality of positions, and after controlling the stirrer such that the difference between the temperatures is reduced, controls the reconstruction processing unit such that the reconstruction processing unit performs the acquisition of the sound speed.

9. The object information acquiring apparatus according to claim 1, further comprising:
a driving mechanism configured to change a relative positional relationship between the conversion element and the object, wherein
the temperature measuring unit is configured to measure the temperatures in accordance with each of the positional relationships changed by the driving mechanism, and
the controlling unit is configured to control the reconstruction processing unit based on information on the temperatures in each of the positional relationships.

10. The object information acquiring apparatus according to claim 1, wherein
the temperature measuring unit is configured to measure each of the temperatures of the acoustic matching member when the reception of the acoustic wave is started and ended, and
the controlling unit is configured to perform estimation processing of the temperature and the sound speed of the acoustic matching member in a time period between the start of the reception of the acoustic wave and the end of the reception of the acoustic wave.

11. The object information acquiring apparatus according to claim 1, wherein
in a case where, based on the information on the temperatures measured at the plurality of positions, a difference between the temperatures is less than a second prescribed value, the controlling unit performs control in which the reconstruction processing unit considers the sound speed constant and acquires the characteristic information.

12. The object information acquiring apparatus according to claim 11, wherein
in a case where, based on the information on the temperatures measured at the plurality of positions, the difference between the temperatures is more than the second prescribed value, the controlling unit estimates a sound speed distribution of the acoustic matching member and uses the estimated sound speed distribution in the acquisition of the characteristic information by the reconstruction processing unit.

13. The object information acquiring apparatus according to claim 1, wherein
the controlling unit is configured to control the reception of the acoustic wave by the conversion element, based on information on the temperatures measured at the plurality of positions.

14. The object information acquiring apparatus according to claim 1, wherein
the controlling unit is configured to control the acquisition of the sound speed by the reconstruction processing unit, based on information on the temperatures measured at the plurality of positions.

15. An information processing method that acquires characteristic information of an object based on an electric signal obtained by receiving, using a conversion element, an acoustic wave that is generated from the object and propagates, the information processing method comprising:
acquiring information on temperatures at a plurality of positions in an acoustic matching member that acoustically matches the object to the conversion element; and
performing control based on the information on the temperatures at the plurality of positions.
